(19) United States
(12) Patent Application Publication
(10) Pub. No.: US 2013/0143897 A1
(43) Pub. Date: Jun. 6, 2013

(54) ORAL CONTROLLED RELEASE PHARMACEUTICAL COMPOSITIONS OF BLONANSERIN

(71) Applicants: Pravin Meghrajji Bhutada, Pune (IN); Ashish Ashokrao Deshmukh, Pune (IN); Sajeev Chandran, Pune (IN); Shirishkumar Kulkarni, Pune (IN)

(72) Inventors: Pravin Meghrajji Bhutada, Pune (IN); Ashish Ashokrao Deshmukh, Pune (IN); Sajeev Chandran, Pune (IN); Shirishkumar Kulkarni, Pune (IN)

(73) Assignee: LUPIN LIMITED, Mumbai, State of Maharashtra (IN)

(21) Appl. No.: 13/816,342

(22) PCT Filed: Aug. 10, 2011
(86) PCT No.: PCT/IB2011/001843

§ 371 Date: Feb. 11, 2013

(30) Foreign Application Priority Data
Aug. 10, 2010 (IN) 891/KOL/2010

(51) Int. Cl. A61K 31/496

(52) U.S. Cl. CPC A61K 31/496
USPC 514/253.04

(57) ABSTRACT

An oral controlled release pharmaceutical composition comprising Blonanserin and release controlling agent(s) and optionally pharmaceutically acceptable excipients is provided. The present invention further relates to a controlled release pharmaceutical composition comprising Blonanserin and release controlling agent(s) such that the composition releases not less than about 80% of Blonanserin within 20 hours, when dissolution is carried out in 900 ml, 0.1 N HCl, USP apparatus Type II (Paddle) at 50 rpm for 20 hrs. The controlled release pharmaceutical composition of the invention releases 50% of Blonanserin between about 4 to 14 hours, when dissolution is carried out in 900 ml, 0.1 N HCl, USP apparatus Type II (Paddle) at 50 rpm.

### FIELD OF THE INVENTION

The present invention relates to oral controlled release pharmaceutical compositions comprising Blonanserin and method for the treatment of disorders such as psychosis or schizophrenia.

### BACKGROUND OF THE INVENTION

Schizophrenia is a serious mental illness characterized with delusion, hallucinations (positive symptoms) as well as asociality, alogia and anhedonia (Negative symptoms). Antipsychotic drugs are the first line therapy for the treatment of schizophrenia.

Blonanserin (2-(4-Ethyl-1-piperazinyl)-4-(4-fluorophenyl)-5,6,7,8,9,10-hexahydrocycloocta[b]pyridine) is an atypical antipsychotic which was disclosed in U.S. Pat. No. 5,021,421. Blonanserin exhibits potent binding property to serotonin (S₂) and dopamine (D₂) receptors, thereby increasing the effect on concentration of brain monoamines metabolites. Blonanserin is used to improve the effect for positive symptoms such as hallucination, delusion etc., negative symptoms such as emotional withdrawal, apathia etc. occurring in schizophrenia.

Blonanserin is commercially available as Lonasen® immediate release tablets 2 mg, 4 mg & 8 mg, and powder 2% strengths in Japan and in South Korea. The approved dosage of Blonanserin includes administration of 4 mg Blonanserin twice daily after meal and dosage can be gradually increased for adults. As continuation dosage, 8˜16 mg daily is administered orally after meal by dividing into 2 times. The dosage may be increased or decreased appropriately however; a single dosage should not exceed 24 mg.

Thus, the dosing regimen recommended for Blonanserin requires frequent administration, in order to attain a constant therapeutic level of Blonanserin, which may lead to poor patient compliance and increases chances of missing the recommended dose. The frequent administration of immediate release tablets leads to fluctuation in drug levels which may further lead to precipitation of adverse effects and therefore this unavoidable fluctuation of drug concentration makes it difficult to attain a steady state condition.

Compared with immediate-release pharmaceutical compositions, controlled-release pharmaceutical compositions provide uniform drug release and can decrease the frequency of administration required to maintain therapeutically effective plasma drug levels. In addition, by producing more constant blood levels, such compositions can reduce the fluctuations in plasma levels observed between doses.

There exists a need to develop pharmaceutical compositions that can alleviate the problems associated with conventional immediate release dosage forms.

The invention provides oral controlled release pharmaceutical compositions of Blonanserin. The compositions provide effective therapeutic concentration of Blonanserin in a controlled manner over a prolonged or extended period of time. Such pharmaceutical compositions also offer other advantages namely increased patient compliance and reduced side effects.

### SUMMARY OF THE INVENTION

In accordance, one embodiment discloses oral controlled release pharmaceutical compositions comprising Blonanserin and release controlling agents.

Another embodiment discloses oral controlled release pharmaceutical compositions comprising Blonanserin and release controlling agents, wherein the release controlling agents are selected from hydrophilic and/or hydrophobic agents.

Yet another embodiment discloses oral controlled release pharmaceutical compositions of Blonanserin wherein the composition releases not less than about 80% of Blonanserin within 20 hours.

Yet another embodiment discloses oral controlled release pharmaceutical compositions of Blonanserin wherein the composition releases about 50% of Blonanserin between 4 to 14 hours.

In a specific embodiment, controlled release pharmaceutical compositions of Blonanserin exhibit substantial bioequivalence to conventional immediate release composition of Blonanserin administered twice daily under fed conditions in a single dose study.

In yet another embodiment, controlled release pharmaceutical compositions of Blonanserin comprising reduced dose of Blonanserin corresponding to dose of immediate release composition of Blonanserin, wherein the compositions exhibit substantial bioequivalence to conventional immediate release composition of Blonanserin under fed conditions at steady state.

Further, embodiment discloses the use of oral controlled release pharmaceutical compositions of Blonanserin for the treatment of psychosis or schizophrenia.

### BRIEF DESCRIPTION OF THE DRAWINGS

FIG. 1: shows a release profile of controlled release pharmaceutical composition of Blonanserin of example 1, in 900 ml of 0.1N HCl, USP apparatus Type II (Paddle) at 50 rpm for 20 hrs.

FIG. 2: represents the dissolution profile of Example XI, Example XII and Example XIII in 900 ml of 0.1 N HCl, USP apparatus Type II (Paddle) at 50 rpm.

FIG. 3: represents the comparative in vivo profile of Example XI, Example XII and reference in a single dose study.

FIG. 4: represents the comparative in vivo profile of Example XIII and reference in steady state for 24 hours

FIG. 5: represents the comparative in vivo profile of Example XIII and reference in steady state from 96-120 hours.

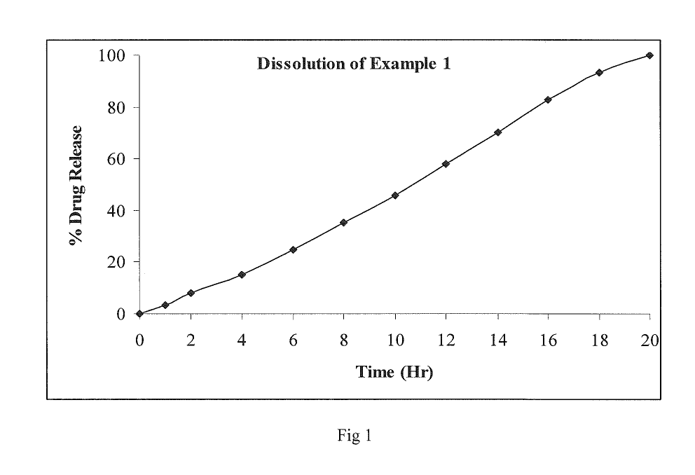

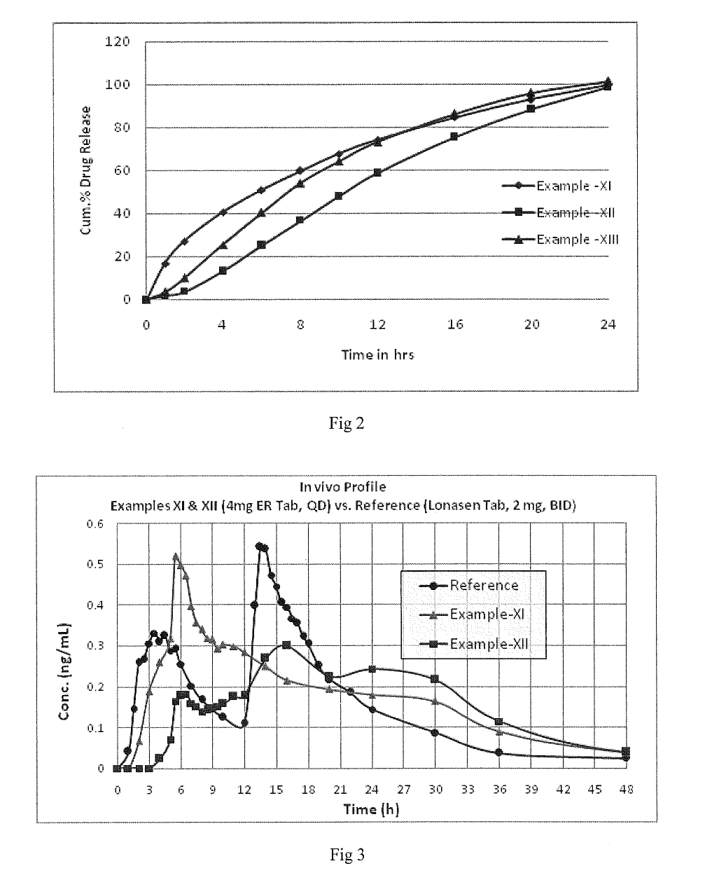

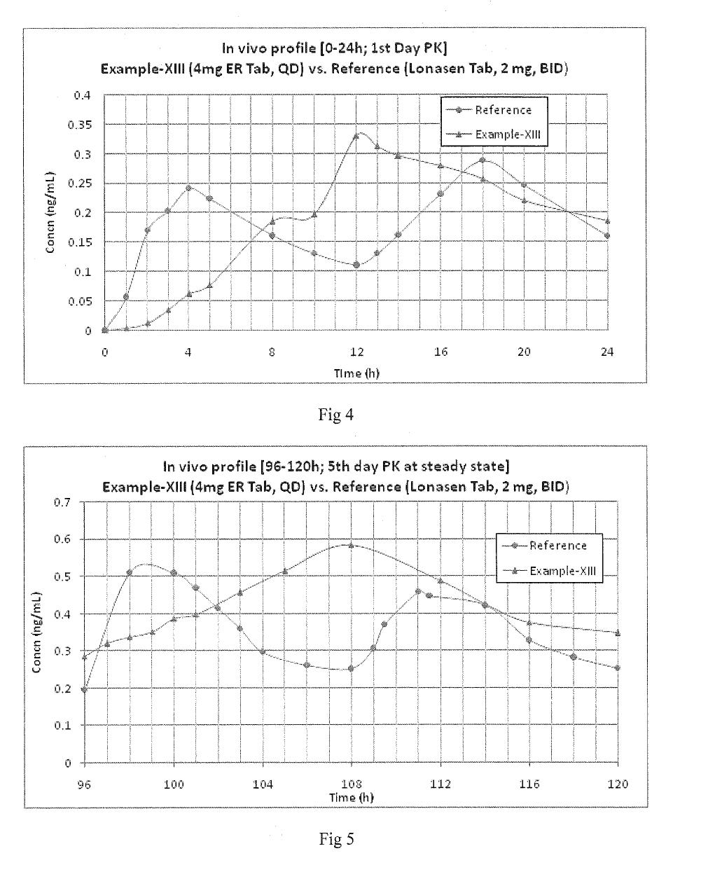

### DETAILED DESCRIPTION OF THE INVENTION

The oral controlled release pharmaceutical compositions of Blonanserin which can deliver Blonanserin in a controlled manner over a period or extended period of time.

The oral controlled release pharmaceutical compositions comprising Blonanserin and release controlling agents.

The amount of Blonanserin to be used ranges from about 0.1 mg to about 24 mg, preferably in the range of about 3 mg to about 16 mg.

As used herein “Blonanserin” encompasses free base, pharmaceutically acceptable salts, pharmacologically active metabolites of Blonanserin and their pharmaceutically acceptable salts, hydrates, its enantiomer or its racemates unless otherwise noted.

The pharmaceutically acceptable salts include but are not limited to inorganic acids (e.g. hydrochloride, hydrobromide, hydroiodide, sulfate, phosphate, etc.), and salts of organic acids (e.g. maleate, fumarate, citrate, oxalate, tartrate, lactate, benzoate, methanesulfonate, etc.). Preferably Blonanserin is used as a free base.

Blonanserin exhibit poor solubility in water. Such poorly water soluble drug result in lower dissolution and absorption which in turn affect drug bioavailability. Particle size reduction of poorly soluble drugs improves the dissolution rate which provides better absorption. Therefore, the compositions may contain Blonanserin in micronized form. The term “micronized” used herein refers to effective particle size of Blonanserin is below 100 microns. The term “effective particle size” used herein refers to D₉₀ particle size of Blonanserin is less than about 100 microns. In a more preferred embodiment D₉₀ particle size of Blonanserin is less than about 50 microns. In the most preferred embodiment D₉₀ particle size of Blonanserin is less than about 20 microns.

Particle size reduction and the measurement of particle size can be done by various techniques known in the art.

The controlled-release pharmaceutical compositions comprising Blonanserin and pharmaceutically acceptable salts thereof, and release controlling agents and optionally pharmaceutically acceptable excipients.

The release controlling agents may be selected from hydrophilic release controlling agents, hydrophobic release controlling agents, or mixtures thereof.

The hydrophilic release controlling agents are selected from, but are not limited to, hydroxypropyl methyl cellulose (HPMC), hydroxypropyl cellulose (HPC), hydroxyethyl cellulose (HEC), polyethylene oxide, polyvinyl alcohol, polyvinylpyrrolidone, xanthan gum, guar gum, chitosan and its derivatives, carbomer, carrageenan, carboxymethyl cellulose, sodium alginate, polyglycolized glycerides, polyethyleneglycol, or mixture thereof.

The hydrophobic release controlling agents are selected from, but are not limited to, polyvinyl acetate dispersion, ethyl cellulose, cellulose acetate, cellulose propionate (lower, medium or higher molecular weight), cellulose acetate propionate, cellulose acetate butyrate, cellulose acetate phthalate, cellulose triacetate, poly (methyl methacrylate), poly (ethyl methacrylate), poly (butyl methacrylate), poly (isobutyl methacrylate), and poly (hexyl methacrylate), poly (isodecyl methacrylate), poly (lauryl methacrylate), poly (phenyl methacrylate), poly (methyl acrylate), poly (isopropyl acrylate), poly (isobutyl acrylate), poly (octadecyl acrylate), waxes such as beeswax, carnauba wax, paraffin wax, microcrystalline wax, and ozokerite; fatty alcohols such as cetostearyl alcohol, stearyl alcohol, cetyl alcohol and myristyl alcohol, and fatty acid esters such as glyceryl monostearate; glycerol monooleate, acetylated monoglycerides, tristearin, tripalmitin, cetyl esters wax, glyceryl palmitostearate, glyceryl behenate, or hydrogenated vegetable oils.

The amount of the release controlling agent may range from about 5% to about 95% by weight of the composition. Preferably from about 25% to about 75% by weight of the composition and more preferably from about 35% to about 65% by weight of the composition.

The pharmaceutical compositions of Blonanserin are prepared using one or more release controlling agent(s) being present either in the core, or in the coating layer. Alternatively the release controlling agent(s) can be present in both the core and the coating layer(s).

The term “controlled release pharmaceutical compositions” herein refers to any composition which comprises Blonanserin, which is formulated to provide a gradual release of Blonanserin over a relatively longer period of time so that the concentration of Blonanserin is maintained in the blood for a longer time at a more uniform concentration than a corresponding immediate release composition comprising the same drug in the same amount. Controlled release pharmaceutical compositions mean any pharmaceutical composition which is other than immediate release pharmaceutical composition or is exchangeable with for example, extended release, sustained release, delayed release, controlled release or pulsed-release at a particular time.

As used herein, the term “bioequivalence” denotes a scientific basis on which two or more pharmaceutical compositions containing same active ingredient are compared with one another. “Bioequivalence” means the absence of a significant difference in the rate and extent to which the active agent in pharmaceutical equivalents or pharmaceutical alternatives becomes available at the site of action when administered in an appropriately designed study. Bioequivalence can be determined by an in vivo study comparing a pharmacokinetic parameter for the two compositions. Parameters often used in bioequivalence studies are Cmax, AUC_{0-inf} and AUC₀₋ₜ. In the present context, substantial bioequivalence of two compositions is established by 90% confidence intervals (CI) of between 0.80 and 1.25 for AUC.

A single dose study is a study in which a controlled release product of interest is given to patients only once, and its corresponding immediate release reference listed drugs are dosed for an equivalent 12 hour dose as directed by the approved label on the immediate release reference listed drugs. The controlled release pharmaceutical composition exhibits a mean Cₘₐₓ in the range of about 0.15 to 0.9 ng/ml, preferably in the range of about 0.25 to about 0.6 ng/ml in the single dose study. The controlled release pharmaceutical composition exhibits a mean AUC(0-t) in the range of about 4.4872 to 8.9622 ng/ml*h, preferably in the range of about 3.0821 to about 5.7239 ng/ml*h in the single dose study.

A steady state study is a study in which the controlled release product of interest and immediate release reference listed drugs are given repeatedly over time during the study until a steady-state blood serum level of the drugs are achieved. The phrase “Cₘₐₓ SS” refers to the highest serum concentration (e.g., ng/ml) observed in a patient on repeated administration after steady state has been reached. The phrase “Cₘᵢₙ SS” refers to the lowest serum concentration (e.g., ng/ml) observed in a patient after steady state for the drug has been reached. The phrase “C_{avg} SS” refers to the average serum concentration at steady state. The controlled release pharmaceutical composition exhibits a mean Cₘₐₓ in the range of about 0.15 to 1.2 ng/ml, preferably in the range of about 0.4 to about 0.8 ng/ml at steady state. The controlled release pharmaceutical composition exhibits a mean AUC in the range of about 4.4872 to 14.5824 ng/ml*h, preferably in the range of about 7.5050 to about 13.8340 ng/ml*h at steady state.

The controlled release pharmaceutical compositions at reduced dose exhibit substantial bioequivalence to conventional immediate release compositions of Blonanserin at steady state under fed condition. The controlled release pharmaceutical composition exhibits a mean Cₘₐₓ in the range of about 0.15 to 0.9 ng/ml, preferably in the range of about 0.3 to about 0.7 ng/ml at steady state. The controlled release pharmaceutical composition exhibits mean AUC in the range of about 4.4872 to 12.3950 ng/ml*h, preferably in the range of about 5.6288 to about 11.7589 ng/ml*h at steady state.

The term “reduced dose” used herein refers to the 15-25% lower than the dose of Blonanserin used in immediate release compositions. For example; reduced dose of controlled release pharmaceutical composition will be about 1.5 mg to about 1.7 mg with respect to immediate release composition containing 2 mg of Blonanserin. Similarly, reduced dose of controlled release pharmaceutical composition will be about 3.0 mg to about 3.4 mg with respect to immediate release composition containing 4 mg of Blonanserin. Further, reduced dose of controlled release pharmaceutical composition will be about 6.0 mg to about 6.80 mg with respect to immediate release composition containing 8 mg of Blonanserin. Similarly, reduced dose of controlled release pharmaceutical composition will be about 12.0 mg to about 13.60 mg with respect to immediate release composition containing 16 mg of Blonanserin.

In another embodiment controlled release pharmaceutical compositions of Blonanserin exhibit substantial bioequivalence to conventional immediate release composition of Blonanserin under fed conditions at steady state wherein the difference between maximum and minimum concentration at steady state for controlled release pharmaceutical compositions of Blonanserin is lower than the conventional immediate release composition of Blonanserin.

The term “Tₘₐₓ” denotes the time to reach the maximal plasma concentration (Cₘₐₓ) after administration; AUC_{0-inf} or AUC denotes the area under the plasma concentration versus time curve from time 0 to infinity; AUC₀₋ₜ denotes the area under the plasma concentration versus time curve from time 0 to time t. For statistical analysis of pharmacokinetic data, the logarithmic transformed AUC₀₋ₜ, AUC_{0-∞}, or Cₘₐₓ data can be analyzed statistically using analysis of variance.

The term “controlled release pharmaceutical compositions” is not restricted to any particular type of composition. The various types of controlled release pharmaceutical compositions may be used for example, a solid oral pharmaceutical composition that encompasses one or more individual units. The individual units may be in form of granules, pellets, minitablets or beads. Granules, pellets, minitablets or beads can be filled into a capsule, sachet or alternatively compressed into a tablet.

Solid oral pharmaceutical compositions may be prepared by any conventional techniques, not restricted to, dry granulation, direct compression, wet granulation, and extrusion-spheronization, melt granulation or compression coating.

The pharmaceutically acceptable excipients includes, but are not limited to, diluents, binders, solubilizing agents, dissolution enhancing agents, pore forming agents, osmagents, gas forming agents, lubricants and glidants known to person skilled in the art.

Diluents include, but are not limited to, lactose, fructose, dextrose, sucrose, maltose, microcrystalline cellulose, starch, calcium hydrogen phosphate, mannitol or any combinations thereof.

Binders include, but are not, limited to starch, sugars, gums, polyvinylpyrrolidone, low molecular weight hydroxypropyl methylcellulose, hydroxypropyl cellulose, hydroxyethyl cellulose or any combinations thereof.

Solubilizing agents include, but are not limited to, surfactants, cyclodextrin and its derivative, lipophilic substances or any combinations thereof.

Surfactants include, but are not limited to, water soluble or water dispersible nonionic, semi-polar nonionic, anionic, cationic, amphoteric or zwitterionic surface active agents or any combinations thereof.

Examples of nonionic surfactants include, but are not limited to, fatty alcohols such as cetyl alcohol, stearyl alcohol, cetostearyl alcohol (consisting predominantly of cetyl and stearyl alcohols) and oleyl alcohol, polyoxyethylene/polyoxypropylene glycol alkyl ethers (Brij) such as octaethylene glycol monododecyl ether, pentaethylene glycol monododecyl ether, glucoside alkyl ethers such as decyl glucoside, lauryl glucoside, octyl glucoside, polyoxyethylene glycol octylphenol ethers (Triton X-100), polyoxyethylene glycol sorbitan alkyl esters such as Polysorbate, sorbitan alkyl esters such as Span and block copolymers of polyethylene glycol and polypropylene glycol such as Poloxamer.

Non limiting examples of anionic surfactant include ammonium lauryl sulfate, sodium lauryl sulfate, sodium laureth sulfate, sodium myreth sulfate, dioctyl sodium sulfosuccinate.

Cationic surfactants include, but are not limited to, quaternary ammonium cation such as alkyltrimethylammonium salts; cetyltrimethylammonium bromide, cetylpyridinium chloride, benzalkonium chloride and benzethonium chloride.

Amphoteric surfactants include, but not limited to, imidazoline-based and fatty amine-based surfactants. Examples include the imidazoline based amphoteric surfactants such as cocoamidoalkylamino monoacetate, cocoamidoalkylamino monopropionate sodium cocoamidoalkylamino hydroxypropyl sulfonate, sodium caprylamidoalkylamino hydroxypropyl sulfonate, and the fatty alkyl amine-based amphoteric surfactants such as cocoalkylamine acetates, cocoalkylamine diacetates, cocoalkylamine propionates, cocoalkylamine dipropionates, and cocoalkylamine hydroxypropylsulfonates.

Other solubilizing agents include, but are not limited to, vitamin E and its derivatives; monohydric alcohol esters such as trialkyl citrates, lactones and lower alcohol fatty acid esters; nitrogen containing solvents; phospholipids; glycerol acetates such as acetin, diacetin and triacetin; glycerol fatty acid esters such as mono-, di- and triglycerides and acetylated mono- and diglycerides; propylene glycol esters; ethylene glycol esters and combinations thereof.

Dissolution enhancing agents include, but are not limited to, organic acids, inorganic acids or combination thereof. The organic acids include, but not limited to citric acid, fumaric acid, malic acid, maleic acid, tartaric acid, succinic acid, oxalic acid, aspartic acid, mandelic acid, glutaric acid, and glutamic acid. The inorganic acids include but not limited to hydrochloric acid, phosphoric acid, nitric acid, and sulfuric acid.

Pore formers may include, but are not limited to, alkali metal salts, alkali earth metal salts, transition metal salts, organic compound or combination thereof. The alkali metal salts include sodium chloride, sodium bromide, potassium chloride, potassium sulfate, potassium phosphate, sodium benzoate, sodium acetate, sodium citrate, potassium nitrate, and the like. The alkali earth metal salts include calcium phosphate, calcium nitrate, and the like. The transition metal salts include ferric chloride, ferrous sulfate, zinc sulfate, cupric chloride, manganese fluoride, manganese fluorosilicate, and the like. The pore formers include organic compounds such as polysaccharides.

Osmagents include, but are not limited to, magnesium sulfate, magnesium chloride, sodium chloride, lithium chloride, potassium sulfate, sodium carbonate, sodium sulfite, lithium sulfate, potassium chloride, sodium sulfate, d-mannitol, urea, sorbitol inositol, raffinose, sucrose, glucose, mixtures thereof, and the like. The osmagent is usually present in an excess amount, and it can be in any physical forms, such as particle, powder, granule, and the like.

Solid gas forming agents include, but are not limited to, suitable carbonate, such as calcium carbonate, sodium carbonate or sodium hydrogen carbonate, with sodium hydrogen carbonate being preferred. Liquid gas-forming agents may be methyl formate, tetramethyl silane, iso-pentane, isomers of perfluoropentane, diethyl or diethenyl ether. The gas-forming agent may be in combination with said matrix, or directly or indirectly affixed thereto.

Lubricants include, but are not limited to, talc, magnesium stearate, calcium stearate, stearic acid, sodium stearyl fumarate, sodium benzoate and the like.

Glidants include, but are not limited to, colloidal silicon dioxide, talc and the like.

In an embodiment Blonanserin and release controlling agents are mixed with suitable pharmaceutical excipients and the blend is granulated with a suitable solvent optionally containing release controlling agents. The granulated blend is dried and further mixed with suitable pharmaceutical excipients. This blend can be filled into capsules or can be compressed into tablets.

In another embodiment Blonanserin, suitable pharmaceutical excipients and optionally release controlling agents are granulated by techniques known in the art. A coating comprising release controlling agents is applied onto the granules which can be filled into capsules or can be compressed into a tablet.

In one embodiment, controlled release pharmaceutical compositions can be formulated in the form of osmotic tablets that can be produced, including a semi permeable membrane as a coating on one or more segments of the dosage form or containing an exit orifice. The segments of the tablet can be coated after compression. The purpose of the membrane is to allow fluids in, thus allowing the typically inactive layer to swell, creating an osmotic gradient against the active layer.

The osmotic tablet may be in the form of a two-layer tablet with a drug and push layer. The drug layer comprises Blonanserin, the release controlling agents and optionally suitable pharmaceutical excipients. The push layer is constructed of release controlling agent(s) and an osmagent. When the composition comes in contact with an aqueous environment, the lower compartment swells and pushes against the diaphragm. Consequently, the upper chamber contracts, thereby delivering the drug through the orifice as a solution or a suspension.

Orifice used herein comprises means and methods suitable for releasing the active ingredient or drug from the osmotic system. The orifice can be formed by mechanical drilling, laser drilling or by eroding an erodible element such as a gelatin plug in the environment of use.

In another embodiment, controlled release pharmaceutical compositions can be formulated in the form of mucoadhesive matrix system wherein the active ingredient or drug is dissolved and/or dispersed in the polymer matrix system with selective, high efficacy delivery to specific regions of the gastrointestinal tract, which includes drug to be delivered, release controlling agents, and one or more bioadhesive agents. The bioadhesive agents may be either dispersed in the matrix of the solid oral dosage form or applied as a direct compressed coating to the solid oral dosage form. The compression coating can be applied to one of the face or multiple face of the tablet.

As used herein “mucoadhesive” generally refers to the ability of a material to adhere to a biological surface for an extended period of time.

Mucoadhesive agents are hydrophobic enough to be non-water-soluble, but contain a sufficient amount of exposed surface carboxyl groups to promote adhesiveness. These include, among others, non-water-soluble polyacrylates and polymethacrylates; polymers of hydroxy acids, such as polylactide and polyglycolide; polyanhydrides; polyorthoesters; blends comprising these polymers; and copolymers comprising the monomers of these polymers.

Optionally, the mucoadhesive agents are a blend of hydrophilic agents and mucoadhesive hydrophobic agents.

In another embodiment, controlled release pharmaceutical compositions can be formulated in the form of gastroretentive dosage form. The gastroretentive dosage form can be prepared either of employing the basic principles such as Swelling and Expanding Systems, Floating and Buoyancing System, Bioadhesive System, Ion Exchange Resin, Magnetically Controlled Gastric Residence and the like.

Another embodiment provides oral controlled release pharmaceutical compositions of Blonanserin wherein the compositions release not less than about 80% of Blonanserin within 20 hours. In a preferred embodiment, oral controlled release pharmaceutical compositions of Blonanserin release about 50% of Blonanserin between 4 to 14 hours.

A suitable dissolution test is where the measurement is carried out in a type II dissolution (50 rpm) apparatus according to U.S. pharmacopoeia in 0.1 N HCl at 37° C. for 20 hours or variations on this as well known to one who is skilled in the art.

Another embodiment provides the use of an oral controlled release pharmaceutical composition(s) comprising rate controlling agent(s) for the treatment of a disorder or disease such as psychosis and schizophrenia.

The controlled-release pharmaceutical composition comprising Blonanserin and release controlling agent(s) which is substantially bioequivalent to conventional immediate release composition of Blonanserin (Lonasen® 2 mg) administered twice daily. Following two studies were conducted to assess the substantial bioequivalence of the compositions of the invention.

Study 1:

This study was carried out to compare the rate and extent of absorption of single dose of two test controlled release pharmaceutical compositions (Example XI and Example XII) of Blonanserin administered once daily with Lonasen® 2 mg tablet administered twice daily (one tablet every 12 hour) under fed conditions.

Study 2:

This study was performed to compare the rate and extent of absorption of repeated administration of test controlled release pharmaceutical composition (Example XIII) of Blonanserin administered once every 24 hour with Lonasen® 2 mg tablet administered twice daily (one tablet every 12 hour) under fed conditions for 5 days.

### Study 1:

Study 1 was an Open label, balanced, randomized, three treatment, three-sequence, three-period, single dose, crossover bioequivalence study which was performed in 8 healthy, adult, male, human volunteers who meet all inclusion under standard fed conditions. Following 3 treatments were given to the volunteers for the purpose of the study:

1) Reference [Lonasen® Tablet 2 mg administered twice daily]
2) Example-XI [Blonanserin ER Tab 4 mg administered once daily]
3) Example-XII [Blonanserin ER Tab 4 mg administered once daily]
Following Pharmacokinetic Parameters of Reference and Test (Example XI and Example XII) were Compared:

Primary parameters: Cₘₐₓ, AUC₍₀₋ₜ₎ and AUC_{(0-inf)}

### Results of Substantial Bioequivalence Study:

TABLE 1
Represents comparative pharmacokinetic parameters of composition of Example XI
and Example XII with reference (Lonasen ®):

| | | | | Relative Bioavailability | |
|---|---|---|---|---|---|
| Test Parameters | | Test | | (% T/R) | |
| Mean | Reference | Example XI | Example XII | Example XI | Example XII |
| Cₘₐₓ (ng/ml) | 0.518 | 0.459 | 0.337 | 88.6 | 65.1 |
| AUC₍₀₋ₜ₎ | 6.246 | 6.894 | 6.401 | 110.4 | 102.5 |
| (ng/ml * h) | | | | | |
| AUC_{(0-inf)} | 7.132 | 8.719 | 0.337 | 122.3 | 109.4 |
| (ng/ml * h) | | | | | |

Cₘₐₓ = Maximum plasma concentration
AUC₍₀₋ₜ₎ = Area under the plasma concentration v. Time curve from 0 hours to the time of last sample collected, t = 72 hours
AUC_{(0-inf)} = Area under the plasma concentration v. Time curve from 0 hours to infinity.

### Study 2

Study 2 was an Open label, balanced, randomized, two treatment, two-sequence, two-period, crossover, first day and steady state (5^{th} day) bioequivalence study which was performed in healthy, adult, male, human volunteers who meet all inclusion under standard fed conditions.

Following 2 treatments were given to the volunteers for the purpose of the study:

1) Reference [Lonasen® Tablet 2 mg administered twice daily for 5 days]
2) Example-XIII [Blonanserin ER Tab 4 mg administered once daily for 5 days]

Table 2a and 2b:

represents comparative pharmacokinetic parameters of composition of Example XIII with reference (Lonasen®) on 1^{st} day and 5^{th} day (steady state).

TABLE 2a
(Day 1)
Example XIII vs Reference (0-24 hours) 1st day Pharmacokinetic
Parameter Data in 10 and 9 volunteers for reference and test
respectively

| | | | Relative |
|---|---|---|---|
| Test Parameters | | | Bioavailability |
| Mean | Reference | Example XIII | (% T/R) |
| Cₘₐₓ (ng/ml) | 0.341 | 0.365 | 107.2 |
| AUC₍₀₋₂₄₎ (ng/ml * h) | 4.424 | 4.403 | 99.9 |
| Tₘₐₓ | 13.1* | 11.2 | |

*Based on 2^{nd} dose

TABLE 2b
(Day 5)
Example XIII vs Reference (96-120 hours) 5th day Pharmacokinetic
Parameter Data in 9 and 7 volunteers for reference and test
respectively

| | | | Relative |
|---|---|---|---|
| Test Parameters | | | Bioavailability |
| Mean | Reference | Example XIII | (% T/R) |
| Cₘᵢₙ SS (ng/ml) | 0.184 | 0.254 | 133.2 |
| Cₘₐₓ SS (ng/ml) | 0.696 | 0.736 | 106.1 |
| Difference (Cₘₐₓ − Cₘᵢₙ) | 0.512 | 0.482 | — |
| C_{avg} SS (ng/ml) | 0.357 | 0.434 | 121.6 |
| AUC₍₉₆₋₁₂₀₎ (ng/ml * h) | 8.602 | 10.416 | 121.9 |

It must be noted that as used in the specification and the appended claims, the singular forms also include plural unless the context clearly dictates otherwise.

The following non-limiting examples illustrate the various embodiments of the present invention.

### Example I

| | |
|---|---|
| Ingredients | Quantity % w/w |

First Layer

| | |
|---|---|
| Blonanserin | 0.5-10 |
| Polyvinyl pyrrolidone | 2-10 |
| Poloxamer | 0.5-10 |
| Hydroxy propyl methyl cellulose | 15-60 |
| Lactose monohydrate | 5-30 |
| Polyethylene oxide | 0-15 |
| Micro crystalline cellulose | 5-30 |
| Magnesium stearate | 0.5-2 |
| Alcohol/dichloromethane mixture | q.s. |

Second Layer

| | |
|---|---|
| Lactose monohydrate | 5-20 |
| Hydrogenated vegetable oil | 2-20 |
| Polyethylene oxide | 2-10 |
| Hydroxy propyl methyl cellulose | 2-20 |
| Silicon dioxide | 0.5-2 |
| Magnesium stearate | 0.5-1 |
| Opadry coat | 2-5% of core weight |

### Brief Manufacturing Procedure:

### First Layer

1) Sift and mix hydroxy propyl methyl cellulose, polyethylene oxide, lactose monohydrate and microcrystalline cellulose in rapid mixer granulator.
2) Dissolve Blonanserin, poloxamer and polyvinyl pyrrolidone in alcohol/dichloromethane mixture.
3) Granulate mixture of step 1 with solution of step 2.
4) Dry the wet granules and sift through suitable sieve.
5) Lubricate the granules using magnesium stearate.

### Second Layer

6) Sift and mix lactose monohydrate, hydrogenated vegetable oil, polyethylene oxide, hydroxy propyl methyl cellulose and silicon dioxide.
7) Lubricate the blend of step 6 using magnesium stearate.
8) Compress the lubricated blend of step 5 and step 7 into bilayer tablets using suitable shaped punches and dies.
9) Coat the tablets using Opadry coat.

### Example II

| Ingredients | Quantity % w/w |
|---|---|
| Blonanserin | 10.0 |
| Hydroxy propyl cellulose | 4.0 |
| Poloxamer | 5.0 |
| Hydroxy propyl methyl cellulose | 30.0 |
| Hydrogenated vegetable oil | 15.0 |
| Lactose monohydrate | 19.0 |
| Microcrystalline cellulose | 15.0 |
| Colloidal silicon dioxide | 1.0 |
| Magnesium stearate | 1.0 |
| Alcohol/dichloromethane mixture | q.s. |
| Ammonio methacrylate copolymer- Type A | 5.0 |
| Ammonio methacrylate copolymer- Type B | 5.0 |
| Triethyl citrate | 1.0 |
| Isopropyl alcohol/acetone mixture | q.s. |

### Brief Manufacturing Procedure:

1) Sift microcrystalline cellulose and lactose monohydrate through 40 # SS sieve.
2) Dissolve Blonanserin, poloxamer and hydroxy propyl cellulose in alcohol/dichloromethane mixture.
3) Granulate the dry mix of step 1 using solution of step 2 in rapid mixer granulator. Dry the wet granules and sift the dried granules through 25 # SS sieve.
4) Mix the dried granules of step 3 with hydroxy propyl methyl cellulose and hydrogenated vegetable oil and colloidal silicon dioxide.
5) Lubricate the granules of step 4 using magnesium stearate.
6) Compress the lubricated blend using suitable tooling.
7) Coat the compress tablets using solution of ammonio methacrylate copolymer-type A, ammonio methacrylate copolymer-type B and triethyl citrate in isopropyl alcohol/acetone mixture.

### Example III

| Ingredients | Quantity % w/w |
|---|---|
| Blonanserin | 5.0 |
| Low substituted hydroxy propyl cellulose | 10.0 |
| Hydroxy propyl methyl cellulose | 40.0 |
| Polyvinyl acetate | 15.0 |
| Lactose monohydrate | 15.0 |
| Microcrystalline cellulose | 14.0 |
| Magnesium stearate | 1.0 |
| Purified water | q.s. |
| Opadry coat (non-functional) | For 2-5% wt. gain |

### Brief Manufacturing Procedure:

1) Sift the Blonanserin, microcrystalline cellulose and lactose monohydrate through 40 # SS sieve.
2) Granulate the above blend using aqueous solution of low substituted hydroxy propyl cellulose as binder.
3) Dry the wet granules and sift the dried granules through 20 # SS sieve.
4) Mix the dried granules of step 3 with hydroxy propyl methyl cellulose and polyvinyl acetate.
5) Lubricate the granules of step 4 using magnesium stearate.
6) Compress the lubricated blend using suitable size & shape punch.
7) Coat the compressed tablets using Opadry coating dispersion.

### Example IV

| Ingredients | Quantity % w/w |
|---|---|
| Blonanserin | 10.0 |
| Polysorbate | 2.0 |
| Polyvinyl pyrrolidone | 7.5 |
| Lactose monohydrate | 42.5 |
| Microcrystalline cellulose | 25.0 |
| Magnesium stearate | 1.0 |
| Ethyl cellulose | 10.0 |
| Polyethylene glycol | 2.0 |
| Ethanol | q.s. |
| Isopropyl alcohol/dichloromethane/acetone mixture | q.s. |

### Brief Manufacturing Procedure:

1) Sift microcrystalline cellulose and lactose monohydrate through 40 # SS sieve and mix in rapid mixer granulator.
2) Dissolve Blonanserin, Polysorbate and polyvinyl pyrrolidone in ethanol.
3) Granulate the blend of step 1 using solution of step 2.
4) Dry the wet granules and sift the dried granules through 25 # SS sieve.
5) Lubricate the granules using magnesium stearate and compress the lubricated blend using suitable sized & shaped punch.
6) Coat the compressed tablets using solution of ethyl cellulose and polyethylene glycol in isopropyl alcohol/dichloromethane/acetone mixture.

### Example V

| Ingredients | Quantity % w/w |
|---|---|
| Blonanserin | 5.0 |
| Polyvinyl pyrrolidone | 5.0 |
| Sodium lauryl sulfate | 5.0 |
| Mannitol | 45.0 |
| Lactose monohydrate | 10.0 |
| Microcrystalline cellulose | 10.0 |
| Magnesium stearate | 1.0 |
| Purified water | q.s. |
| Cellulose acetate | 15.0 |
| Polyethylene glycol | 3.0 |
| Triacetin | 1.0 |
| Acetone | q.s. |

### Brief Manufacturing Procedure:

1) Sift all the ingredients separately with suitable sieve.
2) Mix and granulate Blonanserin, mannitol and lactose monohydrate using aqueous solution of polyvinyl pyrrolidone.
3) Dry the wet granules of step 2 and sift the dried granules through 25 # SS sieve.
4) Blend the dried sifted granules with microcrystalline cellulose, sodium lauryl sulfate.
5) Lubricate the above blend with magnesium stearate.
6) Compress the lubricated blend using round shaped punches of suitable size.
7) Dissolve cellulose acetate in acetone along with triacetin and polyethylene glycol under stirring.
8) Coat the compressed tablets of step 6 using coating solution of step 7.
9) Drill the tablets with tablet laser driller machine to form suitable size orifice.

### Example VI

| | |
|---|---|
| Ingredients | Quantity % w/w |

Hydrophobic polymer granules

| | |
|---|---|
| Blonanserin | 2.5 |
| Hydrogenated vegetable oil | 10.0 |
| Polyethylene glycol | 2.0 |
| Stearic acid | 8.5 |
| Lactose monohydrate | 13.0 |
| Colloidal silicon dioxide | 1.0 |
| Magnesium stearate | 0.5 |

Hydrophilic polymer granules

| | |
|---|---|
| Blonanserin | 2.5 |
| Hydroxy propyl methyl cellulose | 15.0 |
| Xanthan gum | 15.0 |
| Lactose | 20.0 |
| Povidone | 1.5 |
| Lactose monohydrate | 8.5 |
| Colloidal silicon dioxide | 1.0 |
| Magnesium stearate | 0.5 |
| Alcohol/Dichloromethane mixture | qs |
| Opadry coat (nonfunctional) | 2-5% of core |
| | weight |

### Brief Manufacturing Procedure:

### 1) Hydrophobic Polymer Granules

1.1) Sift all ingredients separately through suitable sieve.
1.2) Melt hydrogenated vegetable oil, stearic acid and polyethylene glycol in preheated steam jacketed vessel at 60-70° C. Stop the heating and to this melted mass add Blonanserin under stirring, and continue the stirring for 30-45 min until a uniform mass is formed.
1.3) Cool the molten uniform mass to room temperature and mill the solidified mass in co-mill using suitable sieve.
1.4) Sift the granules of Step 1.3 through suitable sieve.
1.5) Mix the granule of step of 1.4 in suitable blender with lactose monohydrate and silicon dioxide.
1.6) Lubricate the blends of step 1.5 using magnesium stearate.

### 2) Hydrophilic Polymer Granules

2.1) Mix Blonanserin with lactose, hydroxy propyl methyl cellulose and xanthan gum, and granulate with povidone solution in alcohol/dichloromethane mixture.
2.2) Dry the granule of step 2.1 and sift it through suitable sieve.
2.3) Mix the granule of step of 2.2 in suitable blender with lactose monohydrate and silicon dioxide.
2.4) Lubricate the blend of step 2.3 using magnesium stearate
5) Compress the two lubricated blend of step 1.6 and step 2.4 into bilayer tablets using suitable dies and punches.
7) Coat the compressed tablets using Opadry coat.

### Example VII

| | |
|---|---|
| Ingredients | Quantity % w/w |

Active Layer

| | |
|---|---|
| Blonanserin | 5.0 |
| Hydroxymethyl cellulose | 10.0 |
| Mannitol | 20.0 |
| Polyethylene oxide | 15.0 |
| Magnesium stearate | 0.5 |
| Isopropyl alcohol/Dichloromethane mixture | q.s. |

Push-pull layer

| | |
|---|---|
| Polyethylene oxide | 15.0 |
| Potassium chloride | 15.0 |
| Hydroxymethyl cellulose | 3.0 |
| Hydroxy propyl cellulose | 4.0 |
| Magnesium stearate | 0.5 |

Coating (5-15% wt.gain)

| | |
|---|---|
| Cellulose acetate | 10.0 |
| Polyethylene glycol | 1.0 |
| Triacetin | 1.0 |
| Acetone | q.s. |

### Brief Manufacturing Procedure:

### 1) Active Layer

1.1) Sift and mix Blonanserin, with mannitol and polyethylene oxide.
1.2) Granulate the above mixture with binder solution of hydroxymethyl cellulose in isopropyl alcohol/dichloromethane mixture.
1.3) Dry the wet granules of step 1.2 and sift through suitable sieve.
1.4) Lubricate the granules of step 1.3 using Magnesium stearate.

### 2) Push-Pull Layer Granules of Core

2.1) Sift and mix polyethylene oxide, potassium chloride, hydroxymethyl cellulose and hydroxy propyl cellulose.
2.2) Lubricate the above blend with magnesium stearate.
3) Compress granules of step 1.4 and 2.2 as bilayer tablet using suitable size and shape punch.
4) Dissolve cellulose acetate in acetone along with triacetin and polyethylene glycol under stirring.
5) Coat the compressed bilayer tablets of step 3 using coating solution of step 4.
6) Drill the coated tablets with laser drilling technology to form suitable size orifice.

### Example VIII

| Ingredients | Quantity % w/w |
|---|---|
| Blonanserin | 5.0 |
| Poloxamer | 5.0 |
| Hydroxymethyl cellulose | 4.0 |
| Non-peril seed (Sugar sphere) | 39.0 |
| Hydroxymethyl cellulose | 15.0 |
| Alcohol/dichloromethane mixture | q.s. |
| Ethyl acrylate and methyl methacrylate | 30.0 |
| copolymer dispersion | |
| Triethyl citrate | 2.0 |

Brief Manufacturing Procedure:
1) Dissolve Blonanserin, poloxamer and hydroxymethyl cellulose in alcohol/dichloromethane mixture.
2) Coat the above solution on sugar sphere using fluidized bed drier.
3) Coat the above drug loaded pellets with hydroxymethyl cellulose solution in Alcohol/dichloromethane mixture.
4) Coat the above drug loaded seal coated pellets with Ethyl Acrylate and Methyl Methacrylate Copolymer Dispersion along with triethyl citrate.
5) Fill the pellets in suitable capsules or compressed into tablets.

### Example IX

| Ingredients | Quantity % w/w |
|---|---|
| Blonanserin | 5.0 |
| Poloxamer | 5.0 |
| Copovidone | 15.0 |
| Polyethylene glycol | 7.0 |
| Polysorbate 80 | 5.0 |
| Hydroxymethyl cellulose | 40.0 |
| Lactose monohydrate | 20.0 |
| Colloidal silicon dioxide | 2.0 |
| Magnesium stearate | 1.0 |

### Brief Manufacturing Procedure:

1) Melt poloxamer, copovidone and polyethylene glycol at 60-80° C. to it add Blonanserin and polysorbate 80 and stir till solid mass is formed.
2) Pass the above solid mass through co-mill and to it add hydroxymethyl cellulose, lactose and colloidal silicon dioxide and mix well.
3) Lubricate the above blend using magnesium stearate.
4) Compress the above blend using suitable tooling.

### Example X

| Ingredients | Quantity % w/w |
|---|---|
| Blonanserin | 5.0 |
| Low substituted hydroxy propyl cellulose | 10.0 |
| Hydroxy propyl methyl cellulose | 40.0 |
| Polyvinyl acetate | 15.0 |
| Fumaric acid | 2.0 |
| Lactose monohydrate | 15.0 |
| Microcrystalline cellulose | 14.0 |
| Magnesium stearate | 1.0 |
| Purified water | q.s. |
| Opadry coat (non-functional) | For 2-5% wt. gain |

### Brief Manufacturing Procedure:

1) Sift the Blonanserin, microcrystalline cellulose and lactose monohydrate through 40 # SS sieve.
2) Granulate the above blend using aqueous solution of low substituted hydroxy propyl cellulose as binder.
3) Dry the wet granules and sift the dried granules through 20 # SS sieve.
4) Mix the dried granules of step 3 with hydroxy propyl methyl cellulose, polyvinyl acetate and fumaric acid.
5) Lubricate the granules of step 4 using magnesium stearate.
6) Compress the lubricated blend using suitable size & shape punch.
7) Coat the compressed tablets using Opadry coating dispersion.

### Example-XI

| | | |
|---|---|---|
| Sr No | Ingredients | % w/w |

Intragranular

| | | |
|---|---|---|
| 1 | Blonanserin | 0.67 |
| 2 | Citric acid | 0.33 |
| 3 | Microcrystalline cellulose | 27.5 |
| 4 | Lactose monohydrate | 33.3 |
| 5 | Hydroxypropyl methylcellulose | 33.3 |
| 6 | Hydroxypropyl cellulose EXF | 3.33 |
| 7 | Ethanol | qs |
| 8 | Water | qs |

Extra-granular

| | | |
|---|---|---|
| 9 | Colloidal Silicon Dioxide | 0.5 |
| 10 | Magnesium Stearate | 1.0 |

| | |
|---|---|
| Total | 100 |

### Brief Manufacturing Procedure:

1. Dissolve Blonanserin and citric acid in mixture of ethanol and water.
2. Sift the weighed quantity of microcrystalline cellulose, lactose monohydrate, hydroxypropyl methylcellulose and hydroxypropyl Cellulose through 40# SS sieve.
3. Load the blend of step 2 in rapid mixer granulator and dry mix it for 3 min at impeller slow speed.
4. Granulate the blend of step 2 using solution of step 1.
5. Wet mill the granule through suitable sieve.
6. Dry the wet granules of step 5 till its LOD reaches 3.0% w/w.
7. Pass the dried granules of step 6 through suitable sieve.
8. Sift the extra-granular quantity of Colloidal Silicon Dioxide through 40# SS sieve.
9. Mix the granules of step 7 with step 8.
10. Lubricate the blend of step 9 with 60# passed magnesium stearate.
11. Compress the lubricated blend of step 10 using suitable punches and dies.

### Example XII

| | | |
|---|---|---|
| Sr No | Ingredients | % w/w |

Intragranular

| | | |
|---|---|---|
| 1 | Blonanserin | 0.62 |
| 2 | Citric acid | 0.31 |
| 3 | Microcrystalline cellulose | 25.74 |
| 4 | Lactose monohydrate | 30.42 |
| 5 | Hydroxypropyl methylcellulose | 35.10 |
| 6 | Hydroxypropyl cellulose EXF | 3.12 |
| 7 | Ethanol | qs |
| 8 | Water | qs |

Extra-granular

| | | |
|---|---|---|
| 9 | Colloidal silicon dioxide | 1.09 |
| 10 | Magnesium stearate | 1.09 |

Functional Coating

| | | |
|---|---|---|
| 11 | Ethyl cellulose | 1.67 |
| 12 | Hydroxypropyl methylcellulose | 0.62 |
| 13 | Triethyl citrate | 0.20 |
| 14 | Isopropyl alcohol | qs |
| 15 | Dichloromethane | qs |

| | |
|---|---|
| Total | 100 |

### Brief Manufacturing Procedure:

1. Dissolve Blonanserin and citric acid in mixture of ethanol and water.
2. Sift the weighed quantity of microcrystalline cellulose, lactose monohydrate, hydroxypropyl methylcellulose, and hydroxypropyl cellulose through 40# SS sieve.
3. Load the blend of step 2 in rapid mixer granulator and dry mix it for 3 min at impeller slow speed.
4. Granulate the blend of step 3 using solution of step 1.
5. Wet mill the granule through suitable sieve
6. Dry the wet granules of step 5 till its LOD reaches 3.0% w/w.
7. Pass the dried granules of step 6 through suitable sieve.
8. Sift the extra-granular quantity of Colloidal silicon dioxide through 40# SS sieve.
9. Mix the granules of step 7 with step 8.
10. Lubricate the blend of step 9 with 60# passed magnesium stearate.
11. Compress the lubricated blend of step 10 using suitable punches and dies.

### Functional Coating:

12. Dissolve the required quantity of Ethyl cellulose and Hydroxypropyl methylcellulose in isopropyl alcohol & dichloromethane mixture with continuous stirring till the clear solution is formed.
13. Add triethyl citrate to step 12 with continuous stirring. Continue the stirring for further 20 min.
14. Coat the tablets of step 11 using coating solution of step 13 till 2-3% w/w weight gain is obtained using suitable coating parameters.
15. Cure the tablets in coating pan at 50° C. inlet temp for 15-30 min.

### Example-XIII

| | | |
|---|---|---|
| Sr No | Ingredients | % w/w |

Intragranular

| | | |
|---|---|---|
| 1 | Blonanserin | 0.62 |
| 2 | Citric acid | 0.31 |
| 3 | Microcrystalline cellulose | 25.74 |
| 4 | Lactose monohydrate | 30.42 |
| 5 | Hydroxypropyl methylcellulose | 35.10 |
| 6 | Hydroxypropyl Cellulose EXF | 3.12 |
| 7 | Ethanol | qs |
| 8 | Water | qs |

Extra-granular

| 10 | Colloidal silicon dioxide | 1.09 |
|---|---|---|
| 11 | Magnesium stearate | 1.09 |
| | Core Total weight | |

Functional Coating

| | | |
|---|---|---|
| 12 | Ethyl cellulose | 1.56 |
| 13 | Hydroxypropyl methylcellulose | 0.7 |
| 14 | Triethyl citrate | 0.23 |
| 15 | Isopropyl alcohol | qs |
| 16 | Dichloromethane | qs |

| | |
|---|---|
| Total | 100 |

### Brief Manufacturing Procedure:

1. Dissolve Blonanserin and citric acid in mixture of ethanol and water.
2. Sift the weighed quantity of microcrystalline cellulose, lactose monohydrate, hydroxypropyl methylcellulose, and hydroxypropyl cellulose through 40# SS sieve.
3. Load the blend of step 2 in rapid mixer granulator and dry mix it for 3 min at impeller slow speed.
4. Granulate the blend of step 2 using solution of step 1.
5. Wet mill the granule through suitable sieve
6. Dry the wet granules of step 5 in till its LOD reaches 3.0% w/w.
7. Pass the dried granules of step 6 through suitable sieve.
8. Sift the extra-granular quantity of Colloidal silicon dioxide through 40# SS sieve.
9. Mix the granules of step 7 with step 8.
10. Lubricate the blend of step 9 with 60# passed magnesium stearate.
11. Compress the lubricated blend of step 10 using suitable punches and dies.

### Functional Coating:

12. Dissolve the required quantity of ethyl cellulose and hydroxypropyl methylcellulose in isopropyl alcohol & dichloromethane mixture with continuous stirring till the clear solution is formed.
13. Add triethyl citrate to step 12 with continuous stirring. Continue the stirring for further 20 min.
14. Coat the tablets of step 11 using coating solution of step 13 till 2-3% w/w weight gain is obtained using suitable coating parameters.
15. Cure the tablets in coating pan at 50° C. inlet temp for 15-30 min.

1. A controlled release pharmaceutical composition comprising Blonanserin and release controlling agent(s) and optionally pharmaceutically acceptable excipients.

2. The controlled release pharmaceutical composition of claim 1, wherein the release controlling agents are selected from hydrophilic release controlling agents, hydrophobic release controlling agents or mixtures thereof.

3. The controlled release pharmaceutical composition of claim 2, wherein the hydrophilic release controlling agents are selected from hydroxypropyl methyl cellulose, hydroxypropyl cellulose, hydroxyethyl cellulose, polyethylene oxide, polyvinyl alcohol, polyvinylpyrrolidone, xanthan gum, guar gum, chitosan and its derivatives, carbomer, carrageenan, carboxymethyl cellulose, sodium alginate, polyglycolized glycerides, polyethylenglycol, or mixtures thereof.

4. The controlled release pharmaceutical composition of claim 2, wherein the hydrophobic release controlling agents are selected from polyvinyl acetate dispersion, cellulose esters, cellulose ethers and cellulose ester ethers ethyl cellulose, cellulose acetate, cellulose propionate (lower, medium or higher molecular weight), cellulose acetate propionate, cellulose acetate butyrate, cellulose acetate phthalate, cellulose triacetate, poly (methyl methacrylate), poly (ethyl methacrylate), poly (butyl methacrylate), poly (isobutyl methacrylate), and poly (hexyl methacrylate), poly (isodecyl methacrylate), poly (lauryl methacrylate), poly (phenyl methacrylate), poly (methyl acrylate), poly (isopropyl acrylate), poly (isobutyl acrylate), poly (octadecyl acrylate), waxes such as beeswax, carnauba wax, paraffin wax, microcrystalline wax, and ozokerite; fatty alcohols such as cetostearyl alcohol, stearyl alcohol, cetyl alcohol and myristyl alcohol, and fatty acid esters such as glyceryl monostearate; glycerol monooleate, acetylated monoglycerides, tristearin, tripalmitin, cetyl esters wax, glyceryl palmitostearate, glyceryl behenate, and hydrogenated vegetable oils or mixture thereof.

5. The controlled release pharmaceutical composition of claim 1, wherein pharmaceutical acceptable excipients are selected from diluents, binders, solubilizing agents, dissolution enhancing agents, pore forming agents, osmagents, gas forming agents, lubricants, glidants or mixtures thereof.

6. A controlled release pharmaceutical composition comprising Blonanserin and release controlling agent(s) wherein the composition releases not less than about 80% of Blonanserin within 20 hours, when dissolution is carried out in 900 ml, 0.1 N HCl, USP apparatus Type II (Paddle) at 50 rpm for 20 hrs.

7. The controlled release pharmaceutical composition comprising Blonanserin and release controlling agent(s), wherein the composition releases 50% of Blonanserin between about 4 to 14 hours, when dissolution is carried out in 900 ml, 0.1 N HCl, USP apparatus Type II (Paddle) at 50 rpm.

8. A controlled release pharmaceutical composition comprising Blonanserin and release controlling agent(s) wherein the composition exhibits substantial bioequivalence to conventional immediate release composition of Blonanserin administered twice daily under fed conditions in a single dose study.

9. The controlled release pharmaceutical composition of claim 8, wherein the composition exhibits a mean Cmax from about 0.15 to about 0.9 ng/ml.

10. The controlled release pharmaceutical composition of claim 9, wherein the composition exhibits a mean Cmax from about 0.25 to about 0.6 ng/ml.

11. The controlled release pharmaceutical composition of claim 8, wherein the composition exhibits a mean AUC(0-t) from about 4.4872 to about 8.9622 ng/ml*h.

12. The controlled release pharmaceutical composition of claim 11, wherein the composition exhibits a mean AUC(0-t) from about 3.0821 to about 5.7239 ng/ml*h.

13. A controlled release pharmaceutical composition comprising Blonanserin and release controlling agent(s) wherein the composition exhibits substantial bioequivalence to conventional immediate release composition of Blonanserin under fed conditions at steady state.

14. The controlled release pharmaceutical composition of claim 13, wherein the composition exhibits a mean Cmax from about 0.15 to about 1.2 ng/ml.

15. The controlled release pharmaceutical composition of claim 14, wherein the composition exhibits a mean Cmax from about 0.4 to about 0.8 ng/ml.

16. The controlled release pharmaceutical composition of claim 13, wherein the composition exhibits a mean AUC from about 4.4872 to about 14.5824 ng/ml*h.

17. The controlled release pharmaceutical composition of claim 16, wherein the composition exhibits a mean AUC from about 7.5050 to about 13.8340 ng/ml*h.

18. The controlled release pharmaceutical compositions of claim 13 comprising reduced dose of Blonanserin corresponding to dose of immediate release composition of Blonanserin.

19. The controlled release pharmaceutical composition of claim 18, wherein the composition exhibits a mean Cmax from about 0.15 to about 0.9 ng/ml.

20. The controlled release pharmaceutical composition of claim 19, wherein the composition exhibits a mean Cmax from about 0.3 to about 0.70 ng/ml.

21. The controlled release pharmaceutical composition of claim 18, wherein the composition exhibits a mean AUC from about 4.4872 to about 12.3950 ng/ml*h.

22. The controlled release pharmaceutical composition of claim 21, wherein the composition exhibits a mean AUC from about 5.6288 to about 11.7589 ng/ml*h.

23. The use of controlled release pharmaceutical composition of claim 1 for the treatment of psychosis or schizophrenia.
